(19) United States
(12) Patent Application Publication
(10) Pub. No.: US 2026/0191451 A1
(43) Pub. Date: Jul. 9, 2026

(54) WEARABLE MULTI-MODAL SENSING DEVICE FOR REAL-TIME NEUROLOGICAL MONITORING AND DISORDER DETECTION

(71) Applicant: Aishwarya Ramasamy, Louisville, KY (US)

(72) Inventor: Aishwarya Ramasamy, Louisville, KY (US)

(73) Assignee: Aishwarya Ramasamy, Louisville, KY (US)

(21) Appl. No.: 19/012,776

(22) Filed: Jan. 7, 2025

(51) Int. Cl. A61B 5/256; A61B 5/00; A61B 5/0205; A61B 5/145; A61B 5/291; A61B 5/372

(52) U.S. Cl. CPC A61B 5/256; A61B 5/0006; A61B 5/0022; A61B 5/0205; A61B 5/291; A61B 5/372; A61B 5/6803; A61B 5/0077; A61B 5/14542; A61B 5/4094; A61B 5/4818; A61B 2560/0443; A61B 2562/063

(57) ABSTRACT

A wearable neurological monitoring device is disclosed, comprising a modular structure configurable as a headband, wristband, or anklet for placement on various body parts. The device includes a plurality of physiological sensors operable to detect signals such as brain activity, motion, temperature, humidity, sound patterns, and muscle tension, enabling real-time monitoring and diagnosis of neurological conditions like epilepsy, Parkinson's disease, sleep apnea, and stress. A processing unit analyzes sensor data using predefined algorithms to identify patterns indicative of neurological abnormalities and generates diagnostic outputs. The device features a communication module for wireless data transmission to external devices, facilitating remote monitoring and diagnostics. Designed for comfort, adaptability, and energy efficiency, the device supports modular sensor configurations, secure data handling, and compatibility with mobile applications and cloud platforms. This invention addresses the need for accessible, low-cost, and non-invasive neurological health monitoring solutions for diverse environments and populations.

### FIELD OF THE INVENTION

The invention relates to wearable devices incorporating multi-modal sensors for real-time monitoring, analysis, and detection of neurological disorders in diverse environments.

### BACKGROUND OF THE INVENTION

Neurological disorders, such as epilepsy, Parkinson's disease, and sleep apnea, affect millions worldwide, significantly impacting quality of life and requiring timely diagnosis and management. Current monitoring solutions, like electroencephalogram (EEG) systems, are often bulky, expensive, and require expert analysis, making them inaccessible for many, especially in remote or resource-limited settings. These limitations highlight the urgent need for portable, cost-effective, and user-friendly diagnostic tools.

Existing devices primarily rely on single-sensor technologies, limiting their ability to provide comprehensive neurological assessments. Multi-sensor systems exist but are prohibitively expensive or cumbersome for everyday use. Additionally, these systems often lack flexibility, adaptability, and the ability to function effectively outside clinical environments. This gap underscores the necessity of a wearable solution that integrates advanced sensing technologies for accurate, real-time monitoring.

The invention addresses these challenges by incorporating proven, low-cost, multi-modal sensors capable of detecting various neurological parameters, such as EEG activity, body temperature, motion, and acoustic signals. Designed for comfort and versatility, the wearable device can be customized to fit different body parts, enabling continuous, non-invasive monitoring of neurological health in real-world scenarios.

By leveraging advancements in sensing and data analysis technologies, this invention provides a scalable, accessible solution for individuals, healthcare professionals, and organizations. It bridges the gap between complex clinical systems and the growing demand for portable, affordable health monitoring tools, paving the way for improved management of neurological disorders globally.

### BRIEF DESCRIPTION OF THE INVENTION

Aspects and advantages of the invention will be set forth in part in the following description, or may be obvious from the description, or may be learned through practice of the invention.

In one embodiment, the invention comprises a wearable, multi-modal sensing device that integrates various sensors, including EEG, temperature, humidity, accelerometer, gyroscope, piezoelectric, and acoustic sensors. The device is designed to be worn comfortably as a headband or other adaptable form factors, enabling real-time monitoring of neurological health. It utilizes advanced algorithms to analyze sensor data and identify patterns associated with neurological disorders such as epilepsy, Parkinson's disease, sleep apnea, and stress-related conditions. The device processes and displays the data on connected devices like smartphones or computers, facilitating real-time feedback and enabling users to take informed actions. Its modular design allows users to add or remove sensors depending on specific monitoring needs, providing versatility and customization for both individual and professional use.

In another embodiment, the device incorporates wireless connectivity, enabling data transmission to cloud-based systems for remote storage, diagnostics, and sharing with healthcare professionals. This feature is particularly beneficial for remote environments where access to specialized healthcare is limited. The invention's design accommodates alternative power sources, including rechargeable batteries or renewable energy options like solar power, ensuring usability in diverse settings. Additionally, the device's form factor can be adapted for other body parts, such as wrists or ankles, to meet different monitoring requirements. By offering low-cost, portable, and non-invasive solutions, this invention addresses critical challenges in neurological health monitoring, empowering individuals and healthcare professionals to improve the early detection and management of neurological disorders.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 provides a top view of a human head with the location of various sensors in accordance with embodiments of the present disclosure;

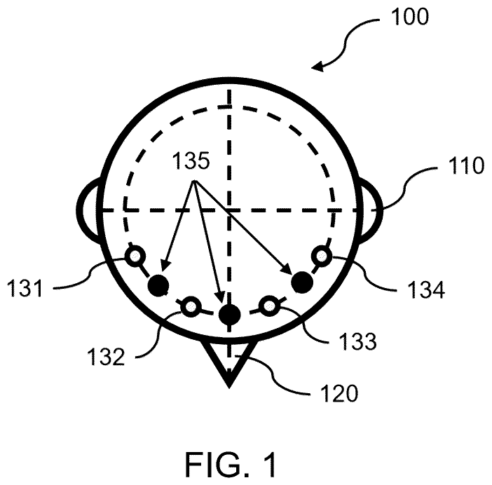

FIG. 2 provides a front view of a human head with the location of various sensors on the forehead in accordance with embodiments of the present disclosure;

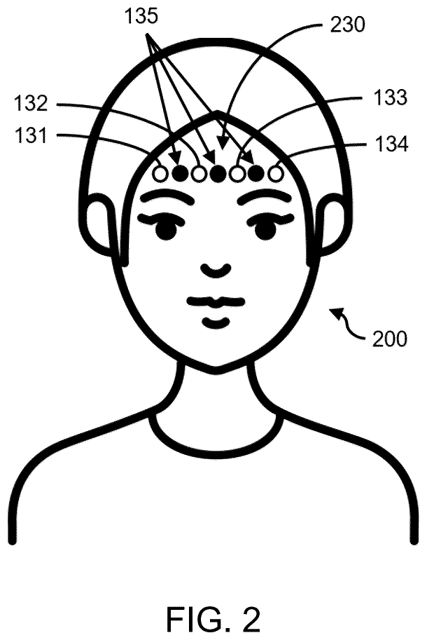

FIG. 3 provides a perspective view of the headband and a human wearing the headband in accordance with embodiments of the present disclosure;

FIG. 4 provides a detailed view of the inner side of the headband with the location of various sensors and the constructional details in accordance with embodiments of the present disclosure;

FIG. 5 provides a data communication system for the headband in accordance with embodiments of the present disclosure;

FIG. 6 provides the electrical control system block diagram of the headband in accordance with embodiments of the present disclosure;

FIG. 7 provides the list of sensors used, its measurements and their applications in accordance with embodiments of the present disclosure;

FIG. 8 provides the list of sensors and its detection of various neurological disorders in accordance with embodiments of the present disclosure;

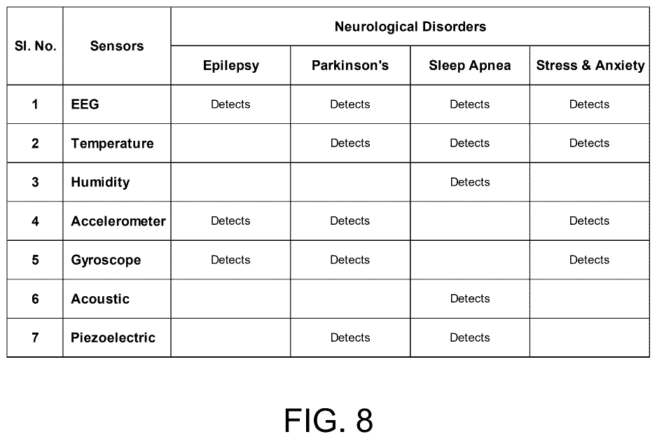

FIG. 9 provides an algorithm flow chart for the possible detection of various neurological disorders using multi-sensors in accordance with embodiments of the present disclosure;

FIG. 10 provides an algorithm for EEG measurement of the headband in accordance with embodiments of the present disclosure;

FIG. 11 provides the epilepsy algorithm for the headband in accordance with embodiments of the present disclosure;

FIG. 12 provides the stress and anxiety algorithm for the headband in accordance with embodiments of the present disclosure;

FIG. 13 provides the sleep apnea algorithm for the headband in accordance with embodiments of the present disclosure;

FIG. 14 provides the Parkinson's disorder algorithm for the headband in accordance with embodiments of the present disclosure;

### DETAILED DESCRIPTION OF THE INVENTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

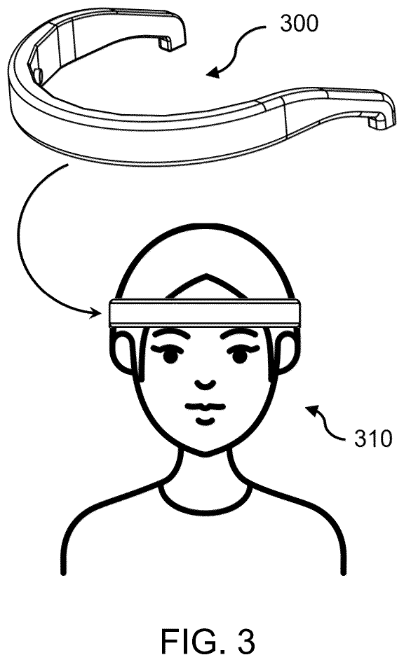

The wearable device 300, as shown in FIGS. 3, 4 and 5, is designed to monitor neurological disorders using multi-modal sensing technologies. It includes various sensors strategically placed to detect physiological parameters. The sensors include an EEG sensor 135, temperature and humidity sensors 134, accelerometer and gyroscope sensors 133, piezoelectric sensors 132, and acoustic sensors 131. These sensors are positioned on a headband to optimize signal accuracy and comfort, as illustrated in FIG. 3.

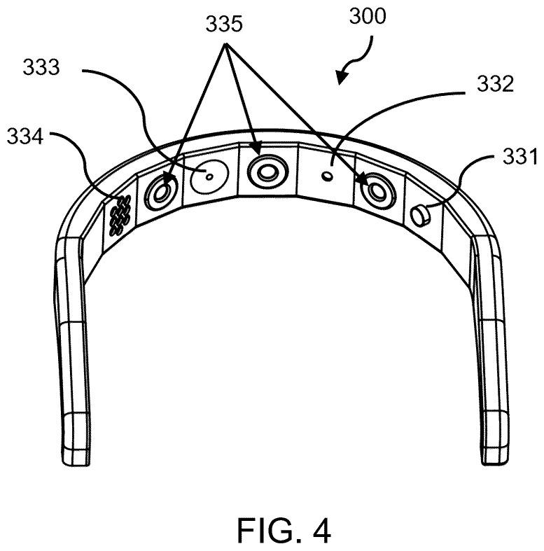

The headband 300 is constructed to ensure flexibility and adaptability. FIG. 4 depicts the inner side of the headband, where the sensors are embedded. Each sensor is mounted in specific locations, such as the forehead 230, to ensure optimal detection of signals. The band itself can be manufactured from materials like plastic, rubber, or metal, as detailed in the features document, to ensure durability and comfort for extended use.

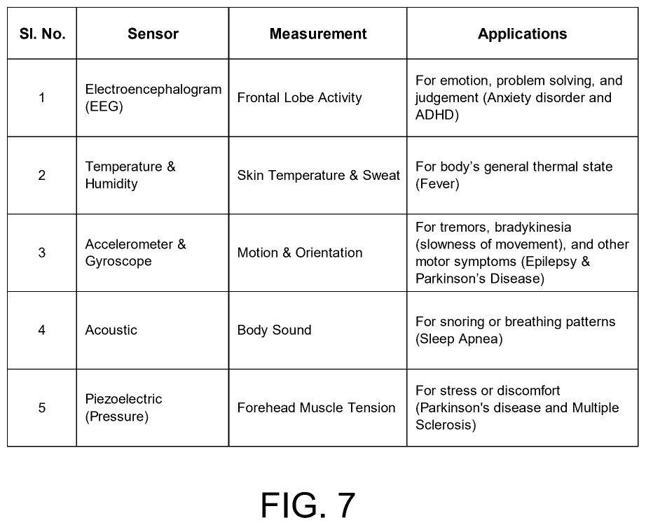

FIG. 7 lists the types of sensors integrated into the device and their respective functions. For instance, the EEG sensor 135 detects brainwave activity to monitor conditions like epilepsy and stress. The temperature and humidity sensors 134 measure thermal states and sweat levels, aiding in detecting stress and fever. The accelerometer and gyroscope sensors 133 analyze motion and orientation, identifying tremors or other motor abnormalities such as those seen in Parkinson's disease. The piezoelectric sensors 132 measure muscle tension, while the acoustic sensors 131 detect breathing patterns for conditions like sleep apnea.

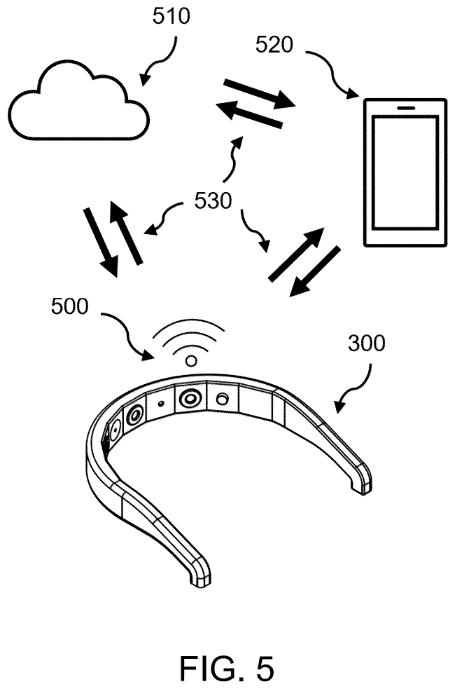

The device is equipped with a data communication system, as shown in FIG. 5, enabling wireless transmission via Wi-Fi, Bluetooth, or SIM card technology. The data is processed and stored either locally or on cloud platforms 510 for remote diagnostics and sharing with healthcare professionals. This enhances the usability of the device in remote and resource-constrained environments.

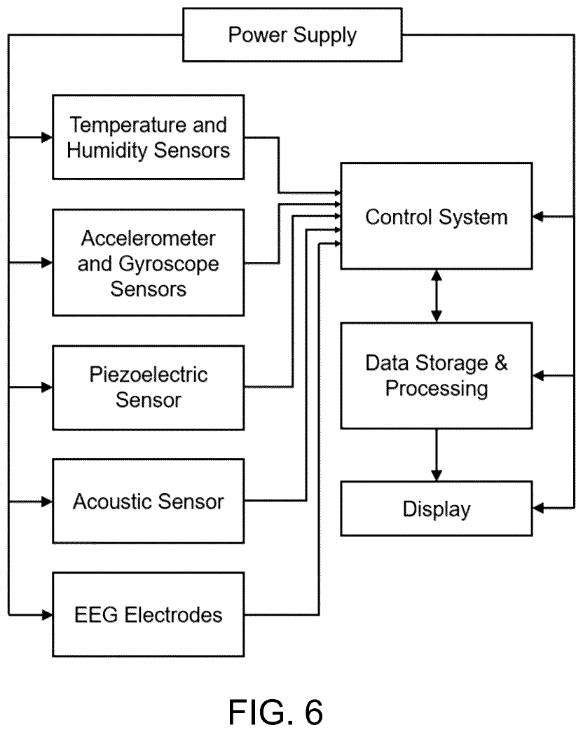

The electrical system of the headband is detailed in FIG. 6, which includes a power supply, a control system, data storage and processing modules, and a display interface. The system integrates algorithms to analyze sensor data in real-time, providing immediate feedback on neurological health.

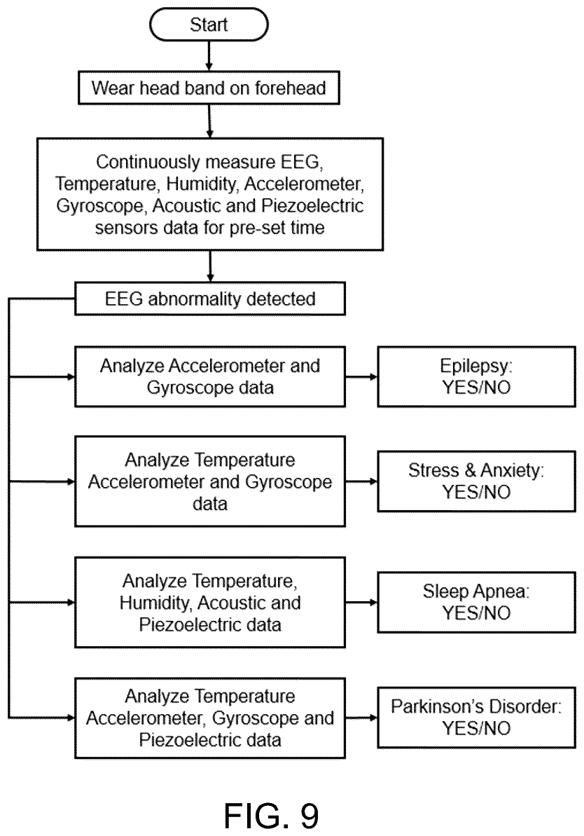

FIG. 9 illustrates the algorithm flowchart used for detecting various neurological disorders. Sensor data is continuously collected, and abnormal patterns are flagged for further analysis. For instance, EEG abnormalities are paired with accelerometer data to detect epilepsy, while temperature and humidity levels combined with acoustic signals identify sleep apnea.

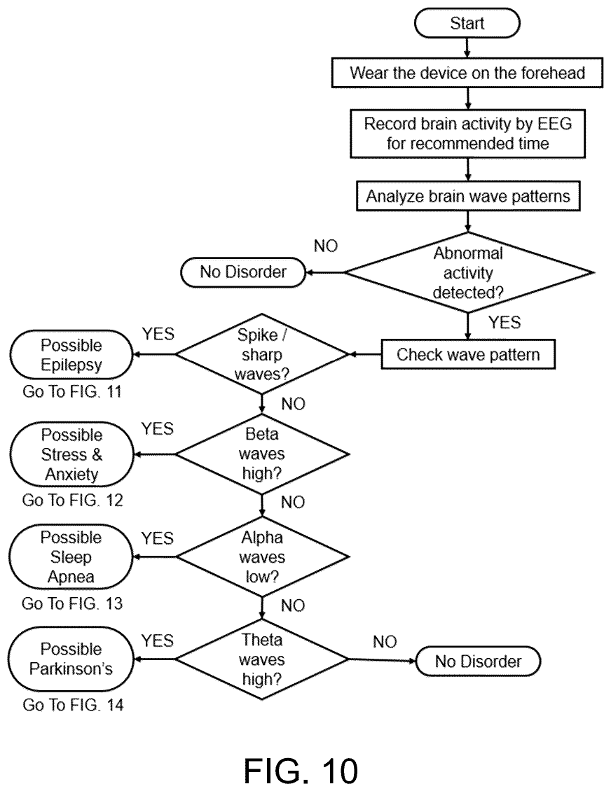

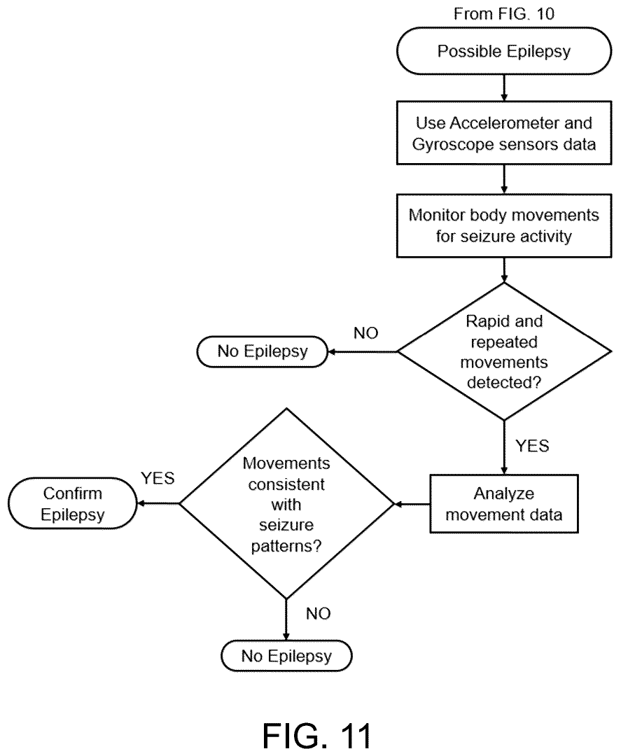

The device incorporates specialized algorithms for EEG analysis, as shown in FIG. 10. Brainwave patterns are categorized into different types, such as beta and alpha waves, to diagnose specific conditions like stress and sleep apnea. FIG. 11 elaborates on the epilepsy detection process, which uses accelerometer data to confirm seizure activity.

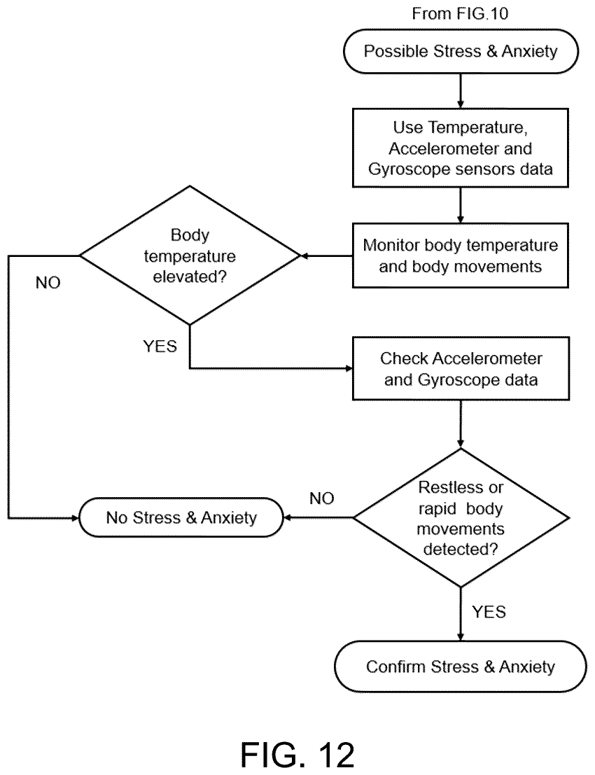

The stress and anxiety detection algorithm is depicted in FIG. 12. It combines body temperature, and motion data to provide an accurate assessment of stress levels. This feature is particularly useful for managing chronic stress-related conditions.

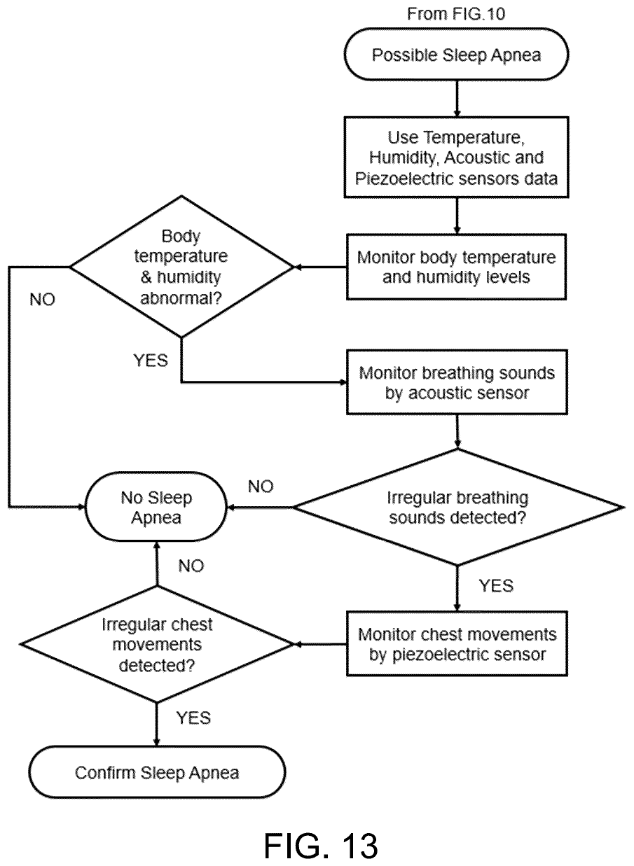

FIG. 13 outlines the diagnostic process for sleep apnea. Abnormal breathing patterns detected by the acoustic sensor 131 and chest movement irregularities identified by piezoelectric sensors 132 are analyzed in real-time, allowing users to monitor sleep health effectively.

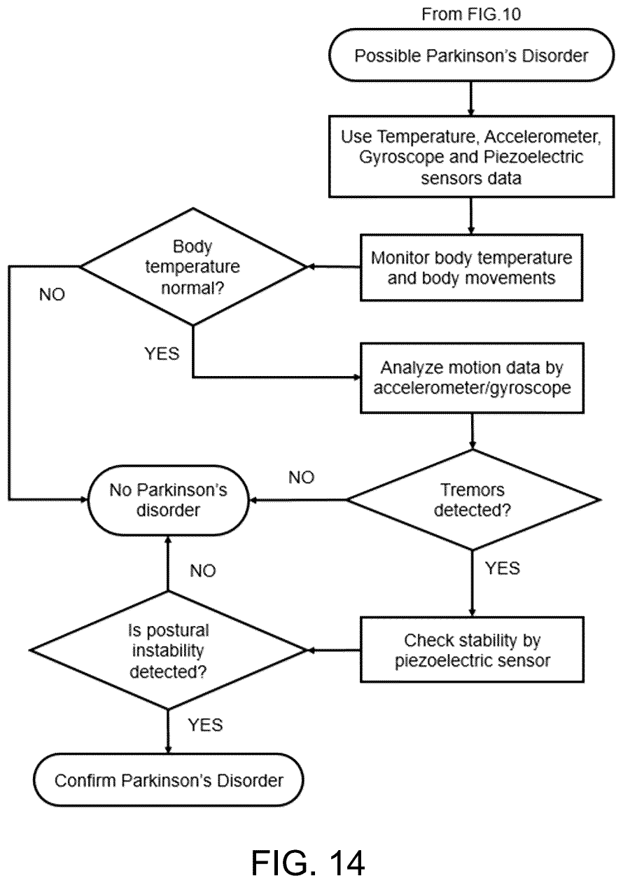

The algorithm for Parkinson's disease diagnosis is shown in FIG. 14. Tremors and postural instability are detected using accelerometer and piezoelectric sensors, offering a non-invasive way to track motor symptoms over time.

The device features real-time feedback mechanisms, displaying results on a connected mobile application or computer interface. FIG. 5 illustrates how this connectivity is established, enabling users and healthcare providers to access and analyze data remotely.

The modular design of the headband allows users to customize the device by adding or removing sensors based on specific needs. For example, acoustic sensors can be excluded for users only interested in monitoring motor-related disorders.

The device supports various power sources, including rechargeable batteries and renewable energy options such as solar panels. This ensures usability in diverse environments, including remote areas without consistent electricity.

The headband can be constructed using rigid, flexible, or semi-flexible materials to suit different user preferences. This adaptability ensures comfort and functionality, even for extended periods of wear.

The wearable device can also be incorporated into other wearable forms, such as wristwatches or anklets, expanding its applications beyond headband usage. This versatility makes the device accessible for various demographic groups, including children and adults.

Multiple prototypes of the device, demonstrate its functionality and effectiveness. The first prototype was wired and tested for basic functionality, while the final prototype was wireless and fully operational, showcasing its advanced capabilities.

The device incorporates secure data encryption for transmitting and storing sensitive health information. This ensures user privacy and compliance with healthcare regulations.

The device is designed for both clinical and non-clinical environments. It can be used by healthcare professionals for diagnostic purposes or by individuals for daily health monitoring, bridging the gap between advanced medical equipment and everyday usability.

The device is scalable for mass production and can be manufactured using cost-effective materials and processes. This ensures affordability, making it accessible to a broader population.

The device supports remote diagnostics, enabling healthcare providers to monitor patients in real-time and offer timely interventions. This feature is particularly valuable in rural and underserved areas.

Additional features, such as integrating cameras or advanced AI algorithms, can further enhance the device's diagnostic capabilities, making it a comprehensive solution for neurological health monitoring.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

### DESCRIPTION OF PART NUMBERS IN THE DRAWINGS

100—Human head top view
110—Human ear
120—Human nose
131—Acoustics sensor
132—Piezoelectric or pressure sensor
133—Accelerometer and gyroscope sensors
134—Temperature and humidity sensors
135—Electroencephalogram (EEG) sensor
200—Human face
230—Human forehead
300—Headband
310—Human wearing headband
331—Acoustics sensor location
332—Piezoelectric or pressure sensor location
333—Accelerometer and gyroscope sensors location
334—Temperature and humidity sensors location
335—Electroencephalogram (EEG) sensors location
500—Wi-Fi or Bluetooth or SIM card communication
510—Cloud
520—Mobile phone
530—Two-way communication

What is claimed is:

1. A wearable neurological monitoring device comprising:
a modular structure configurable as a headband, wristband, or anklet for placement on various parts of the human body;
a plurality of physiological sensors configured to detect signals associated with neurological conditions, wherein the device includes at least one sensor selected from the group comprising an EEG sensor, an accelerometer, a gyroscope, a temperature sensor, a humidity sensor, a piezoelectric sensor, an acoustic sensor, and an oxygen sensor;
a processing unit integrated into the modular structure, the processing unit configured to:
collect signals from two or more physiological sensors;
integrate and analyze the collected signals using predefined multi-sensor algorithms to identify patterns indicative of specific neurological conditions with greater accuracy; and
generate diagnostic outputs based on the analysis;
a communication system operably connected to the processing unit, the communication system configured to wirelessly transmit the diagnostic outputs to external devices for real-time monitoring and further analysis;
wherein the device further comprises a modular sensor interface allowing for the addition or removal of sensors without disrupting device functionality, enabling customization for monitoring specific neurological conditions;
and wherein the integration of oxygen sensor data with EEG and accelerometer data enhances diagnostics for conditions such as epilepsy and sleep apnea.

2. The device of claim 1, wherein the processing unit combines EEG sensor data and oxygen sensor data to improve diagnostics for neurological conditions involving respiratory abnormalities.

3. The device of claim 1, further comprising a rechargeable power supply integrated into the wearable structure, configured to provide continuous operation for at least 12 hours.

4. The device of claim 1, wherein the wearable structure is adjustable to accommodate various body sizes and shapes for improved comfort and fit.

5. The device of claim 1, further comprising a mobile application operable to display real-time diagnostic outputs, send alerts, and provide health recommendations based on integrated sensor data.

6. The device of claim 1, wherein the communication system supports Wi-Fi, Bluetooth, and cellular network connectivity for enhanced data transfer options.

7. The device of claim 1, further comprising a cloud-based platform for storing and analyzing data remotely, with access permissions for healthcare professionals.

8. The device of claim 1, wherein the diagnostic outputs include visual, auditory, or haptic feedback to alert users of detected neurological abnormalities.

9. The device of claim 1, wherein the processing unit is configured to execute algorithms for detecting epilepsy, Parkinson's disease, sleep apnea, stress, and anxiety disorders.

10. The device of claim 1, wherein the wearable structure is constructed from flexible and hypoallergenic materials to ensure comfort and compatibility with extended wear.

11. A wearable device for detecting and diagnosing neurological conditions, comprising:
a structure wearable on various body parts;
at least one physiological sensor selected from the group comprising an EEG sensor, an accelerometer, a gyroscope, a temperature sensor, a humidity sensor, a piezoelectric sensor, an acoustic sensor, and an oxygen sensor, wherein the sensor is operable to detect signals indicative of neurological activity;
a control unit operably connected to at least two of the physiological sensors, the control unit configured to:
collect signals from the two or more sensors;
process and integrate the combined signals to detect abnormalities associated with neurological conditions using predefined adaptive algorithms;
store the processed data locally; and
transmit the processed data wirelessly;
a communication module configured to enable wireless data transfer to external devices for remote monitoring and diagnostics;
wherein the device includes an adaptive power management system that optimizes energy consumption based on active sensors and processing requirements, thereby extending operational battery life during continuous monitoring sessions;
and wherein the integration of sensor outputs from multiple physiological modalities provides enhanced accuracy and insights into conditions such as stress, Parkinson's disease, and respiratory abnormalities.

12. The device of claim 11, wherein the control unit processes combined signals from an accelerometer and gyroscope to detect tremors and postural instability associated with Parkinson's disease.

13. The device of claim 11, wherein the integration of temperature and acoustic sensor data enhances diagnostics for sleep apnea.

14. The device of claim 11, further comprising interchangeable sensors capable of detecting additional physiological parameters, such as blood oxygen levels, heart rate variability, or respiration patterns.

15. The device of claim 11, wherein the control unit supports data encryption to ensure secure transmission and storage of sensitive health information.

16. The device of claim 11, further comprising an integrated camera operable to capture contextual data related to the user's environment for enhanced diagnostic accuracy.

17. The device of claim 11, wherein the processed data is stored locally on the wearable device and transmitted wirelessly to a mobile application or computer interface for further analysis.

18. The device of claim 11, wherein the modular structure includes snap-fit components, enabling quick and easy reconfiguration between different wearable forms such as headbands, wristbands, or anklets.

19. The device of claim 11, further comprising a detachable memory module for exporting recorded data to external storage or diagnostic systems.

20. The device of claim 11, wherein the communication module supports two-way communication, allowing real-time interaction between users and healthcare professionals for remote diagnostics.
